(19) United States
(12) Patent Application Publication
(10) Pub. No.: US 2015/0328322 A1
(43) Pub. Date: Nov. 19, 2015

(54) DRUG DELIVERY VEHICLES

(71) Applicant: THE BOARD OF REGENTS OF THE UNIVERSITY OF TEXAS SYSTEM, Austin, TX (US)

(72) Inventor: Philip SERWER, San Antonio, TX (US)

(21) Appl. No.: 14/653,023

(22) PCT Filed: Dec. 19, 2013
(86) PCT No.: PCT/US2013/076738

§ 371 Date: Jun. 17, 2015

Related U.S. Application Data
(60) Provisional application No. 61/739,718, filed on Dec. 19, 2012.

(51) Int. Cl. A61K 47/42; A61K 31/7048; A61K 31/155; C07K 14/005; C12N 7/00

(52) U.S. Cl. CPC A61K 47/42; C07K 14/005; C12N 7/00; A61K 31/155; A61K 31/7048; C12N 2795/10242; C12N 2795/10222

(57) ABSTRACT

Embodiments are directed to compositions and methods related to delivery of drugs via a capsid shell.

This Application claims priority to U.S. Provisional Application 61/739,718 filed on Dec. 19, 2012, which is incorporated herein by reference in its entirety.

This invention was made with government support under GM24365 awarded by the National Institutes of Health. The United States government has certain rights in the invention.

### BACKGROUND

Embodiments of this invention are directed generally to the fields of biology and medicine. Certain aspects are directed to delivery vehicle for therapeutics.

Cancerous neoplasms caused about 13% of all deaths worldwide in 2007, this percentage is expected to increase as the average age of the World's population increases (Jemal et al., Global cancer statistics. CA: A Cancer Journal for Clinicians 2011, 61:69-90). The work on anti-cancer drug delivery vehicles is currently focused primarily on liposomes, polymeric micelles, silicon nano-particles, and non-silicon inorganic delivery vehicles. These vehicles depend on either the chemistry of their production or attachment of an antibody for specificity (Cobleigh et al., Sem. Oncol. 2003, 30 (5 Suppl 16):117-124; Serda et al., Biochim. Biophys. Acta 2011, 1810:317-329; Chen, Trends Mol. Med. 2010, 16:594-602). In addition, the surface properties of these vehicles vary among particles in a single preparation. Thus, the effectiveness is not as high as it can be because of the non-uniformity of vehicle specificity and inflexibility of surface properties, which cannot be rapidly and flexibly altered by directed evolution to respond to adversities such as (1) undesirable background targeting of healthy cells, (2) clearing of the vehicle by the immune system, and (3) mutation of neoplastic cells that produces resistance to the vehicle. Thus, there remains a need for additional drug delivery vehicles, preferably vehicles encoded by DNA, rather than RNA, in the interest of genomic stability and preferably vehicles that (a) can be diffusion-loaded with multiple drugs, using procedures that increase the internal drug concentration, (b) are highly stable, especially to enzymes, such as proteases and (c) have a gate that is separate from the drug-enclosing shell and can be opened to load drug, closed to transport drug and then opened again to deliver the drug.

### SUMMARY

Certain embodiments are directed drug delivery vehicles that are (1) produced by biological systems in a form that is capable of being loaded and sealed and (2) are capable of optimization for specificity. In certain aspects, optimization can include mutation and/or selection of the drug delivery vehicle. The drug delivery vehicles can include rapidly propagating, biological entities such as bacterial viruses (bacteriophages or phages). In certain embodiments the drug delivery vehicles are double-stranded DNA phages. In certain aspects the drug delivery vehicles are capsids of a double-stranded DNA phage, which are highly stable (protease-resistant) with a low-permeability. In certain aspects the capsid comprises a capsid shell comprising gp10 proteins and a portal comprising gp8 proteins. The portal of the capsid can control permeability to compounds. In certain aspects the drug delivery vehicle can be conditionally permeable to compounds with a molecular weight above at least 314 Daltons (Da). Extension of this range is thought to be feasible via genetic alteration of the gp8 protein or gp10 protein. Under certain conditions, a capsid can be impermeable (for decades in certain instances) to, for example, Metrizamide, which has a molecular weight of 789 Da. Thus, after loading, a drug can be sealed or localized in the cavity as a highly stable, drug-loaded capsid. In certain aspects, capsids can be sealed after loading by modifying temperature, solution conditions, or adding a capping moiety.

In further aspects, the capsid shells can be selected for a particular specificity in binding or localization or property, e.g., in certain aspects a capsid can be selected after mutation of a nucleic acid encoding a capsid component. For example, a capsid can be selected for preferential binding or localization to a target cell or target organ, or for a favorable packaging parameter such as permeability or efficiency relative to a particular drug. A selected capsid typically has the advantages of having needed permeability characteristics, high stability, and simplicity/low expense of purification in 10-100 milligram quantities or greater. The use of mutation/selection enables flexibility and adaptability to vehicle-resistant, targeted cells.

In certain aspects, a drug delivery vehicle as described herein can be use in diagnostic methods. For example, a vehicle can be developed that specifically binds or localizes at or in an imaging target. An imaging vehicle/capsid can be loaded with an imaging agent to form an imaging vehicle. The imaging vehicle can be administered to a subject to be imaged and the imaging target identified by detection of the imaging vehicle in the subject.

In certain embodiments, a capsid can be filled with a drug or other molecule to form a drug delivery vehicle/capsid. In certain aspects drug delivery vehicles described herein are homogeneous, highly stable, non-aggregating, and have a high loading-capacity. In certain aspects a drug will have a molecular weight between about 300 and about 700 Da. These characteristics can of course be altered using mutation and selection of the capsid and/or portal proteins.

Embodiments of the invention include universal drug delivery vehicles comprising capsid shells, e.g., bacteriophage capsid shells. In certain aspects the capsid shells are non-infectious, stable, and can be derived from a rapidly propagated phage.

In certain embodiments pathogenic cells can be contacted with drug delivery vehicles described herein. Pathogenic cells include those cells associated with a disease and include, but are not limited to bacteria, fungi, or eukaryotic cells having an aberrant phenotype. Eukaryotic cells having an aberrant phenotype include hyperplastic cells and cancer cells.

In certain aspects a drug can be an inhibitor of therapeutic target, such as proteins and/or enzymes. As used herein, an “inhibitor” is a chemical compound that can reduce the activity or function of a protein directly or indirectly. Direct inhibition can be accomplished, for example, by binding to a protein and thereby preventing the protein from binding an intended target, such as a receptor, or by inhibiting an enzymatic or other activity of the protein, either competitively, non-competitively, or uncompetitively. Indirect inhibition can be accomplished, for example, by binding to a protein's intended target, such as a receptor or binding partner, thereby blocking or reducing activity of the protein.

In certain aspects a subject can be administered an effective amount of a drug delivery vehicle described herein. The term “effective amount” means an amount effective, at dosages and for periods of time necessary, to achieve the desired therapeutic or prophylactic result. For example, an “effective amount” of an anti-cancer agent can result in a decrease in cancer cell growth or, to some extent, the growth of some cancer or tumor cells. The term includes an amount capable of invoking a growth inhibitory, cytostatic and/or cytotoxic effect, including apoptosis of cancer or tumor cells.

In certain aspects compositions described herein can be used in treating pathological conditions such as cancer. Treating or treatment in reference to cancer, means an amount capable of invoking one or more of the following effects: (1) inhibition, to some extent, of cancer or tumor growth, including slowing down growth or complete growth arrest; (2) reduction in the number of cancer or tumor cells; (3) reduction in tumor size; (4) inhibition (i.e., reduction, slowing down, or complete stopping) of metastasis; (5) enhancement of anti-tumor immune response, which may, but is not required to, result in the regression or rejection of the tumor, or (6) relief, to some extent, of one or more symptoms associated with a cancer or tumor. The therapeutically effective amount may vary according to factors such as the disease state, age, sex and weight of the individual and the ability of one or more anti-cancer agents to elicit a desired response in the individual. A “therapeutically effective amount” is also one in which any toxic or detrimental effects are outweighed by the therapeutically beneficial effects.

Other embodiments of the invention are discussed throughout this application. Any embodiment discussed with respect to one aspect of the invention applies to other aspects of the invention as well and vice versa. The embodiments in the Example section are understood to be embodiments of the invention that are applicable to all aspects of the invention.

The terms “inhibiting,” “reducing,” or “prevention,” or any variation of these terms, when used in the claims and/or the specification, includes any measurable decrease or complete inhibition to achieve a desired result.

The use of the word “a” or “an” when, used in conjunction with the term “comprising” in the claims and/or the specification, may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

Abbreviations used herein include: AUC-BD, analytical buoyant density centrifugation; cryo-EM, cryo-electron microscopy; I, fluorescence intensity; MLD, Metrizamide low density; MHD Metrizamide high density; ts, temperature-sensitive; MLD capsid II Metrizamide low density capsid II.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Illustration of T3 and T7 (A) MLD capsid II and (B) phage.

FIG. 2. Illustration of Cryo-EM asymmetric reconstruction of the phage T7 connector.

FIG. 3. Illustrates an AUC-BD in a Nycodenz density gradient. The arrow indicates a short DNA-containing form of MLD capsid II that can be removed in a preparative centrifugation. The remaining MLD capsid II particles are DNA-free.

FIG. 4. Illustrates the formation of an equilibrium peak during AUC-BD.

FIG. 5. Fluorescence (It) vs. time during the binding of bis-ANS. T7wt is wild type T7 phage; T7-C5, LG3 is a T7 mutant with 8.4% of its DNA deleted. The horizontal bar indicates the fluorescence in the presence of bis-ANS and the absence of a sample.

FIG. 6. Quenching of bound bis-ANS fluorescence vs. time during incubation with ethidium bromide. The horizontal bars indicate the fluorescence before ethidium was added.

FIG. 7. Detailed cryo-EM determined structure of MLD capsid II-associated gp10.

FIG. 8. Scheme for producing DNA fragments for (a, b) PCR-directed random mutagenesis of a targeted genome region and (c, d) PCR-generated, primer-specified introducing of a single base pair change. An “x” indicates a mutagenized base pair.

FIG. 9. Illustrates a scheme for selection of phage that either do not or do bind to a cell of unique type. The cell pictured is a bacterial cell. But, this cell could be any cell, neoplastic or not.

### DESCRIPTION

In the past, phage-based drug delivery vehicles have had limited effectiveness for managing neoplasms, in part because no phage display vector or related vesicle could be diffusion-loaded with a drug and then stably sealed for delivery. Sometimes, the phage display vectors were filamentous phages ( fl, fd, M13) (Staquicini et al., Adv. Drug Delivery Rev. 2010, 62:1213-1216; Bradbury, Curr. Protocols Neurosci. 2010, Chapter 5: Unit 5.12; Bar et al., BMC Biotechnol. 2008, 8:37; Suthiwangcharoen et al., NANO Res. 2011, 4:483-493; Kaur et al., J. Nanotechnol. 2012, Article ID 247427; Budynek et al., Arch. Microbiol. 2010, 192:315-320), without space to diffusion-load drugs. So, in a relatively early study, doxorubicin, and hygromycin were covalently conjugated to the major capsid protein of phage M13 for delivery (Bar et al., BMC Biotechnol. 2008, 8:37). The covalent loading required the introduction of a cathepsin-B cleavage site, used for unloading in lysosomes. Cellular uptake to lysosomes of the conjugated phage occurred via receptor-mediated endocytosis, and the uptake was specific for the cell targeted. Targeting was achieved by binding cell-specific antibodies to the phage capsid. The filamentous phage system was inefficient and rigid.

Subsequently, vesicularization of the major M13 capsid protein was performed with a polymer scaffold (Suthiwangcharoen et al., NANO Res. 2011, 4:483-493). During vesicularization, doxorubicin (mw=544) was diffusion-loaded. But, the vesicles were leaky, unstable, prone to aggregate, non-uniform and selective for (folate receptor-rich) cancer cells at a ratio of about 2:1, in spite of extensive covalent derivation of the vesicles with folate. Furthermore, the process of diffusion-loading was complex and there is no provision for rapid modification of binding specificity was provided.

Other studies used ssRNA virus such as MS-2 as a drug delivery vehicle because they are uniform in size and have an external shell structure. However, the single-stranded RNA phage-based DDVs have the following limitations. (1) The genomic basis is single-stranded RNA, with the expectation of less genomic stability than a DNA genome. (2) Remote loading/drug concentration increasing cannot be achieved, thereby eliminating the drug concentration-boosting potential from remote loading; typically about 80 drug molecules are loaded in a shell 27.5 nm in diameter, a molar drug concentration over an order of magnitude lower than for Doxil. (3) Drugs are covalently joined to RNA molecules, thereby generating complexity, a potential source of irreproducibility and problems in unloading. (4) The protein shell is not stable enough to block release at non-target sites and, indeed, proteases can encourage this to happen.

Intact double-stranded DNA phages are uniform in size and have a large internal cavity for drug diffusion-loading. Even without drug loading, some of them (primarily phages T2 and T4) have anti-tumor activity, which is probably derived from stimulation of the immune system and which has been observed since the 1940s (reviewed in Kaur et al., J. Nanotechnol. 2012, Article ID 247427 and Budynek et al., Arch. Microbiol. 2010, 192:315-320). But, double-stranded DNA phages are also susceptible to bursting and expelling of packaged genome and other contents (Ritchie and Malcolm, J. Gen. Virol. 1970, 9:35-43; Huskey, Mol. Gen. Genet. 1973, 127:39-46). A rapidly modifiable vehicle with space for drug diffusion-loading, a natural gate and high stability, low aggregation, low leaking, and high uniformity was needed.

One embodiment described herein includes a Metrizamide low-density (MLD) capsid II delivery vehicle. In certain aspects the delivery vehicle can be used to deliver anti-cancer compounds. This vehicle retains the advantages of a double-stranded DNA phage vehicle, but reduces leaking, in at least some cases to levels below detectable. MLD capsid II is stable to 80° C., is uniform in cryo-electron microscopy (cryo-EM)-determined size to a resolution of about 3.5 Å, has a large (28 nm in radius), loading-ready internal cavity and is insensitive to all proteases tested, including both trypsin and subtilisin, i.e., MLD capsid II is protease resistant. Furthermore, T7 MLD capsid II is not prone to aggregate and adhere non-specifically to surfaces (Fang et al., J. Mol. Biol. 2008, 384:1384-1399; Serwer, J. Mol. Biol. 1980, 138:65-91; Khan et al., Biophys. J. 1992, 63:1286-1292). MLD capsid II can be isolated nucleic acid-free, which reduces the hazard of using nucleic acid-containing vehicles. Efficient procedures exist for rapidly generating and modifying specificity of binding of MLD capsid II.

In the area of permeability, MLD capsid II has favorable properties for developing a “gated” vehicle that does not release its load in the wrong place and, therefore, does not generate off-target toxicity. Specifically, MLD capsid II has (1) low, but not zero, permeability to compounds, such as anti-neoplastic drugs (i.e., it has diffusion-loading capacity) and (2) zero permeability to compounds with molecular weight higher than a critical value (i.e., it has high capacity for tight sealing) (Khan et al., Biophys. J. 1992, 63:1286-1292). The use of diffusion-loading implies that the chemistry of the compound delivered does not influence the type of delivery vehicle needed, which makes possible the simultaneous delivery of several different drugs. Furthermore, fluorescent molecule-loaded, cell specific MLD capsid II can be used in diagnosis by tagging a target (e.g., a neoplastic) cell. By manipulating the permeability and binding specificity, capsid vehicles can be loaded, sealed, and delivered. In certain aspects, the target cell will unload or deliver when MLD capsid II vehicle arrives in the lysosome of the target cell.

Rapid, managed mutation/selection of the delivery vehicles described provides a means to respond to various adversities that can occur during chemotherapy, such as resistance of a target cell to a capsid vehicle. For example, one risk is that MLD capsid II particles are cleared from the human body too rapidly to have a therapeutic effect. This issue can be resolved by either (1) “humanizing” the exposed surface of gp10, possibly via insertion of a peptide at the C-terminus or (2) selecting for mutant phage particles that are not cleared. For strategy (2), the phage will have been selectively mutagenized, before managed selection, at random positions in the encoding region for the C-terminus of gp10. Strategy (2), without selective mutagenesis, has been successful with phage lambda in mice (Merril et al., Proc. Natl. Acad. Sci. USA 1996, 93:3188-3192).

The highest potential for uniformity, flexibility, and effectiveness resides in drug delivery vehicles that are obtained biologically, rather than chemically. Some DNA phages produce an empty capsid that is comprised of a highly stable, protease resistant, low-permeability shell with a portal of non-shell protein that can be used as drug delivery vehicles. Compounds within a defined molecular weight range diffuse through a portal formed in the empty capsid and are retained within the capsid. The capsid itself can be rapidly engineered to target specific bacteria, cells, tumors, and/or organ sites for drug delivery. In certain aspects, specificity of such a drug delivery vehicle can be achieved using a process of rapid (i.e., on the orders of days) laboratory mutation, propagation, and selection. Uniformity of the vehicle is achieved via biological assembly, i.e., by Mother Nature. The resultant vehicles are selected to specifically bind and deliver a payload to a targeted cell, such as a cancer or other disease-causing cell.

The vehicles described herein may have one or more of the following advantages in relation to current drug delivery vehicles, including, but not limited to (1) increased specificity (and, therefore, capacity for use with drugs of increased cell-killing power), (2) reduced clearance by immune systems (and, therefore, longer time of effectiveness), (3) capacity to be rapidly (days) changed to bypass resistance to the vehicle (and, therefore, increased effectiveness in preventing and managing metastasis), (4) gating of drug entry and release, (5) relatively high uniformity of physical properties, including permeability, and (6) relatively low cost and low complexity of production in high purity.

In certain embodiments capsid protein, gp10, of bacterial DNA viruses (phages), T3 and T7 forms the capsid shell. Certain forms of these capsid shells have been called Metrizamide low density, or MLD, capsid II. These latter capsid shells are produced during DNA packaging. MLD capsid II can be isolated via low density (1.086 g/ml) during preparative buoyant density centrifugation in density gradients of either Metrizamide or Nycodenz. The low density is caused by complete impermeability to Metrizamide (mw=789) so that all of the water of the internal cavity acts as water of hydration (Fang et al., J. Mol. Biol. 2008, 384:1384-1399; Serwer, J. Mol. Biol. 1980, 138:65-91).

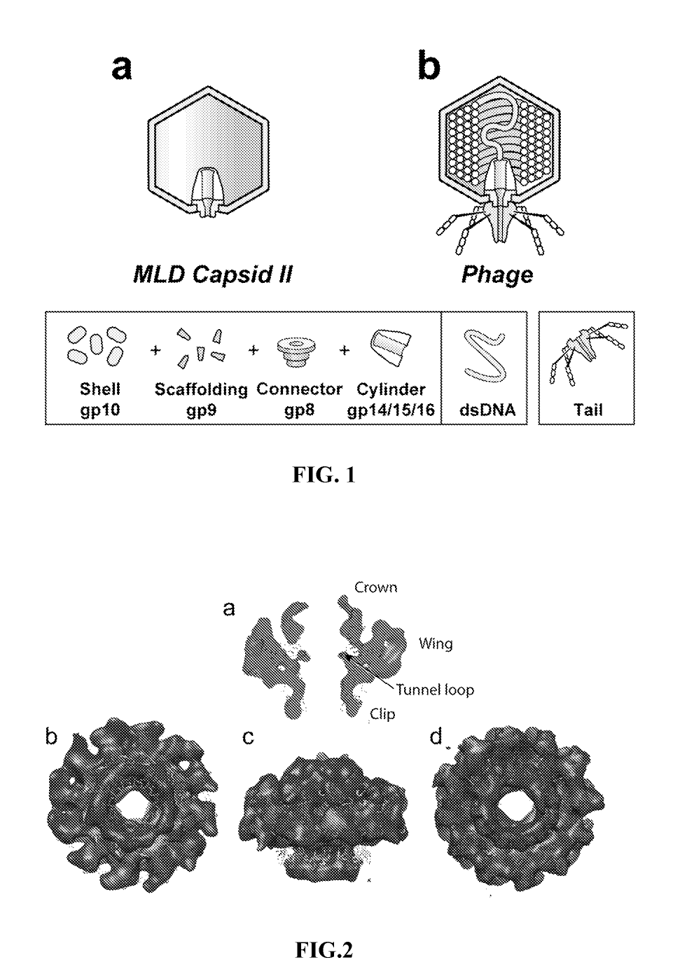

One example of a capsid shell of the invention is the shell of MLD capsid II. MLD capsid II has a shell made of a single protein (called gene product 10, or gp10). A number of gp10 proteins are contemplated for use, for example gp10 proteins of phage T3 (GenBank Accession: KC960671.1 (GI: 505831697)(SEQ ID NO:1)) and phage T7 (GenBank Accession: NC 001604.1 (GI: 9627425)(SEQ ID NO:2)). The phage tail is not present because MLD capsid II appears before a tail is added during assembly. The gp10 is arranged in an icosahedral (T=7) lattice. However, one 5-fold vertex of this gp10 lattice is missing its gp10 pentamer. Instead, a 12-fold ring (“connector”) of a second protein, gp8, is present (FIG. 1a) (Fang et al., J. Mol. Biol. 2008, 384:1384-1399; Serwer, J. Mol. Biol. 1980, 138:65-91; Khan et al., Biophys. J. 1992, 63:1286-1292). The evidence (Khan et al., Biophys. J. 1992, 63:1286-1292) supports the idea that permeability (i.e., capacity for drug loading) of MLD capsid II is normally regulated at the position of the axial channel of the connector when the permeating molecule has a molecular weight of ˜314 Da or higher. Permeability can, in theory, be adjusted either by genetically altering gp8 and/or gp10; or by changing solution conditions.

Most current drug delivery vehicles are limited in their capacity for specificity. Some specificity is obtained via the formation of nano-sized pores in tumor, but not normal vasculature (enhanced permeability and retention effect). In addition, the capsid shell vehicles can be engineered for specificity via mutation/selection of surface peptides or epitopes, which is typically limited by the time needed to propagate and select for production of specific binding characteristics, which with phage is a relatively short time. As a comparison, monoclonal antibody-derived specificity, for example, takes at least two months to achieve (Dreyer et al., BMC Biotechnol. 2010, 10:87; Beck et al., Nat. Rev. Immunol. 2010, 10:345-352). Phage display systems have been developed to more rapidly select for changes in peptides that were inserted in phage capsid proteins; such changes were typically related to binding specificity. The advantage of phage display is that, in a period of days, one selects for a protein with the desired binding specificity and can isolate the gene that encodes the selected protein (Staquicini et al., Adv. Drug Delivery Rev. 2010, 62:1213-1216; Bradbury, Curr. Protocols Neurosci. 2010, Chapter 5:Unit 5.12). Several tumor cell binding-specific peptides have been identified by in vivo selection of phage with displayed peptides and have been used in targeted gene therapy attempts (Bradbury, Curr. Protocols Neurosci. 2010, Chapter 5:Unit 5.12).

For speed of managed mutation/selection, one can use the most rapidly replicating phages, which include the related phages, T3 and T7. These phages replicate so rapidly (a burst of 100-200 generated in 13-15 minutes at 37° C.) that producing specific binding takes little time. A commercial display system for T7 is available and more information can be found on the world wide web at URL merckmilliporechina.com/promart/library/Novagen/5-Novagen-T7Select-Phagedisplay-and-protein-function-research.pdf.

In certain aspects a targeting peptide can be selected by modifying the phage genome by ligating a peptide-encoding DNA segment to the C-terminus-encoding region of gene 10 and, then, packaging the modified genome in vitro, by use of an extract of infected cells (Son et al., Virology 1988, 162:38-46). The C-terminus of gp10 is exposed at the surface of the shell for selection purposes.

In certain aspects the capsid vehicles can be produced and purified in large (10-50 mg) amounts, without using industrial procedures. In certain aspects the capsid vehicles are loaded by diffusion. Use of capsid vehicles also minimizes the non-specific binding of the vehicle to cellular surfaces, which is a problem often caused by the presence of a phage tail (Serwer and Hayes, Electrophoresis 1982, 3:76-80). Non-specific binding can be further minimized by mutation/selection.

One aspect of the capsid vehicle that can be exploited is binding specificity that is generated by more than 2 binding sites present on the capsid shell. For example, the MLD capsid II shell has 415 identical gp10 molecules, of which about 42 are available to bind, for example, a roughly planar segment of cell membrane—as contrasted with an antibody that comprises two binding sites. Thus, a capsid vehicle will potentially bind with greater affinity and specificity given the increased number of binding sites.

A variety of capsids are contemplated for use a capsid vehicle, including, but not limited to T3 and T7 MLD capsid II. T3 MLD capsid II has a surface charge almost 2x that of T7 MLD capsid II (Serwer et al., J. Mol. Biol. 1983, 170:447-469).

In certain aspects a capsid vehicle can be sealed once the capsid vehicle is loaded. Sealing of the capsid vehicle can be accomplished by modification of temperature, solution conditions, modification of the gp8 portal, or addition of a capping moiety.

MLD Capsid II. Studies of permeability indicate that the gp10 shell does not have pores large enough to permit the diffusion-driven permeation of the following dyes: bis-ANS (4,4′-dianilino-1,1′-binaphthyl-5,5′-disulfonic acid; anion mw=597) and ethidium (cation molecular weight=314). Nonetheless, these dyes do diffuse into the internal cavity of MLD capsid II (over hours). Thus, bis-ANS, ethidium, and presumably other similarly sized compounds enter MLD capsid II through the gp8 portal (see FIG. 1a). Metrizamide, which is only slightly larger (mw=789), has never been observed to detectably diffuse into MLD capsid II.

Cryo-EM with asymmetric reconstruction (20 Å resolution) has shown that the connector of T7 has the structure determined for the phage SPP1 connector by x-ray crystallography (Lebedev et al., EMBO J. 2007, 26:1984-1994) at 2Å resolution (FIG. 2). The subunits of the SPP1 connector also have the same mw as the subunits of the T3 and T7 connectors. Thus, the residues in the channel of the connector are the same (±3 residues) for both phages (Guo, F. et al., Proc. Natl. Acad. Sci., USA 2013, 110: 6811-6816).

Cryo-EM with symmetric reconstruction has revealed the structure of the gp10 shells of MLD capsid II and mature T7 phage at 3.2-3.4 Å resolution. These shells have to be homogeneous in structure in order for this reconstruction to have worked (unpublished results of the laboratories of Wen Jiang and Philip Serwer). In addition, a subtle difference, not detected for negatively stained particles, was observed between the MLD capsid II shell and the mature phage shell. First, the MLD capsid II shell was 1.6% (significantly) larger. Second, in relation to the gp10 subunits of the phage shell, the gp10 subunits of the MLD capsid II shell have undergone rigid body rotation around a point on a 5-fold axis. A result is the partial closing of the largest pores in the shell, which are at both true- and pseudo-3-fold axes. The shell of MLD capsid II has a lowered permeability and this permeability can possibly be made either higher or lower by mutating gp10 (unpublished results of the laboratories of Wen Jiang and Philip Serwer).

MLD capsid II is indefinitely stable during storage at either room temperature or 4° C. Preparations left for over 20 years at 4° C. have undergone no change in either shell-connector (portal) structure (based on electron microscopy of a negative stained specimen) or permeability. Compared to preparations of other protein structures, stored MLD capsid II preparations also have a much lower frequency of damage from overgrowth by contaminating environmental bacteria and fungi. Contaminating microbes do not grow well presumably because they have no non-gaseous carbon or nitrogen source in MLD capsid II preparations.

Assays for Loading. To assay for diffusion-driven loading of a compound in the cavity of MLD capsid II, the most direct method is to perform buoyant density equilibrium centrifugation in a non-ionic density gradient of either Metrizamide (mw=789) or the closely related compound, Nycodenz (mw=821). If a compound enters MLD capsid II, the density changes after loading. Most dyes and drugs would, if loaded, cause the density of MLD capsid II to increase. The reason is that most dyes and drugs are denser than water and a loaded dye or drug molecule replaces internal MLD capsid II water. The exceptionally high hydration of MLD capsid II causes the density of MLD capsid II to be extremely low, typically between 1.08 and 1.12 g/ml, decreasing with increasing centrifugation speed, i.e., with increasing pressure (unpublished observations of the laboratories of Borries Demeler and Philip Serwer).

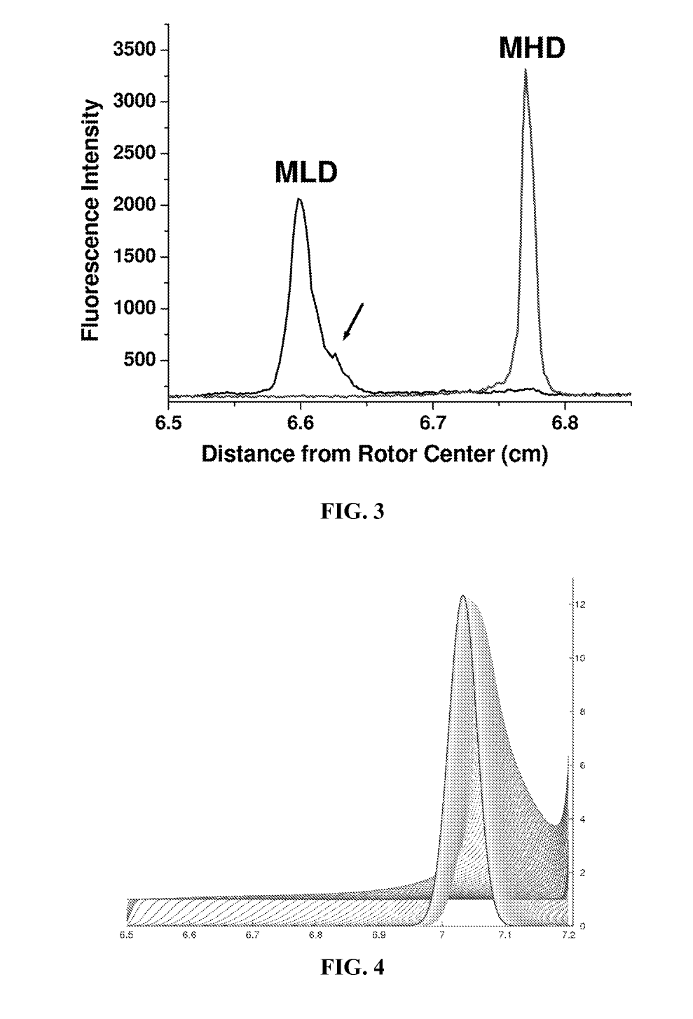

By use of recent technical advances, both densities and their pressure dependence have been explored by use of analytical buoyant density equilibrium sedimentation (AUC-BD) performed with a Nycodenz density gradient and a fluorescence detector. The data were analyzed with the UltraScan-III software (Demeler et al., (2012) UltraScan-III, a comprehensive analysis software for analytical ultracentrifugation experiments. The University of Texas Health Science Center at San Antonio, Dept. of Biochemistry). Sharp peaks were observed in the gradient, yielding high resolution by density (less than 0.002 g/ml resolved). The sources of the peak sharpness are small diffusion coefficient and uniform density of phage and phage capsids. For detection, T7 MLD capsid II was stained with Alexa 488 and followed by AUC-BD (MLD peak in FIG. 3). Fluorescence detection provides picomolar sensitivity and several orders of magnitude of dynamic range. AUC-BD is also efficient in that (1) no gradient fractionation and analysis of separate fractions is needed, (2) sample concentrations in the picomolar range are used, over 2 orders of magnitude less than what is needed for other procedures of detection, (3) 14 samples can be simultaneously run and (4) pressure and temperature dependences are determined with one set of samples by progressively changing the speed and temperature of centrifugation.

The result of loading a compound in MLD capsid II is illustrated by the AUC-BD of a T7 capsid that has the shell of MLD capsid II, but does not have a connector and is, therefore, permeable to Metrizamide and Nycodenz. This latter capsid, called Metrizamide high-density capsid II (MHD capsid II), forms a peak at a density of ˜1.28 g/ml, a density also dependent on hydrostatic pressure (MHD peak in FIG. 3). Detection was by Alexa 488 staining/fluorescence and use of a separate cell in the same rotor as the cell used for MLD capsid II. In certain aspects AUC-BD is used to determine (1) when a compound enters MLD capsid II and (2) the extent to which entry varies among MLD capsid II particles. For example, if some MLD capsid II particles were uniformly permeable to a compound and some were not permeable, then two peaks would form after an attempt at loading MLD capsid II.

The details of the AUC-BD are the following. A sample in buffered Nycodenz is centrifuged to equilibrium. The achieving of equilibrium is judged by the absence of change in peak position. The initial Nycodenz density is controlled by measurement of refractive index. As a Nycodenz density gradient forms, the sample particles migrate to the point at which the density gradient has a density equal to that of the particle (isopycnic density). If the particle is uniform in density and its diffusion is low, then a sharp peak of fluorescence will form. The isopycnic particle density depends on hydration, which in turn varies with both hydrostatic pressure and concentration of Nycodenz at the isopycnic position (Ifft, Meth. Enzymol. 1973, 27:128-140; Hearst et al., Proc. Natl. Acad. Sci. USA. 1961, 47:1015-1025). The effects of pressure result in a dependence of isopycnic density on both centrifugation speed and initial Nycodenz concentration. Pressure depends on the latter via distance from the center of rotation of the centrifuge (Ifft, Meth. Enzymol. 1973, 27:128-140; Hearst et al., Proc. Natl. Acad. Sci. USA. 1961, 47:1015-1025).

The pattern of peak formation is observed by continuous monitoring of fluorescence (FIG. 4). The position of the peak is used to calculate the density of the particle, given the knowledge of centerpiece geometry, meniscus position, rotor speed, molecular weight, partial specific volume, and concentration of the Nycodenz solution, as implemented in UltraScan-III (Demeler et al., (2012) UltraScan-III, a comprehensive analysis software for analytical ultracentrifugation experiments. The University of Texas Health Science Center at San Antonio, Dept. of Biochemistry).

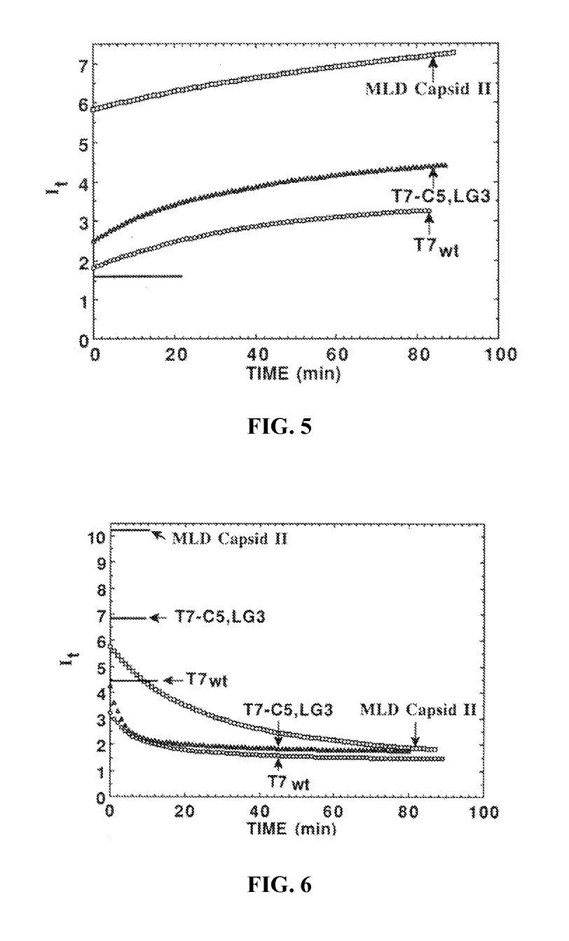

For measuring the rate of loading of a compound in MLD capsid II, an alternative to AUC-BD is usually needed for reasons of efficiency of time and cost. Total fluorescence (/) has been used to determine the rate of loading for bis-ANS, because bis-ANS undergoes fluorescence enhancement when it binds to hydrophobic areas of proteins, including several internal T7 proteins (gps8, gps15, gps16)—the T7 proteins bound were identified by ultraviolet light-induced, covalent cross-linking to bis-ANS after binding. The value of I vs. time at 25° C. is plotted in FIG. 5 (adapted from Khan et al., Biophys. J. 1992, 63:1286-1292), during incubation of bis-ANS with T7 MLD capsid II and also the mature phage T7 and a T7 deletion mutant, C5-LG3. Binding to the exterior of the capsid was so rapid that it was complete before the first measurement. Binding to the interior occurred over several hours, as seen in FIG. 5. Thus, the loading rate is simply observed by measuring I vs. time and subtracting the initial signal. Only rapid (effectively instantaneous) binding occurred in the case of MHD capsid II, a capsid II particle that does not have a connector (not shown here). MLD capsid II-bound bis-ANS fluorescence is quenched by incubation with ethidium cation. This quenching has been used to observe the limited permeability to ethidium of MLD capsid II, phage T7 and the deletion mutant, as illustrated in FIG. 6 (25° C.).

The fluorescence enhancement of MLD capsid II-bound bis-ANS makes possible the use of single-particle fluorescence microscopy to observe single MLD capsid II particles and to observe the distribution of bis-ANS molecules among the MLD capsid II particles (unpublished observation). For this purpose, a previously developed specimen preparation procedure was used that differs from the traditional procedure in that capsids are visualized without attaching them to a solid support and, therefore, without causing attachment-induced distortion of results. Instead, with this procedure, the capsids are allowed to undergo thermal motion, but are confined by extremely narrow boundaries of the zone of buffer. The “narrowly confined buffer” was generated by placing the specimen between a glass microscope slide and a gel that has been cast on a cover glass. This process generates a fluid zone so thin (93-155 nm) that (1) thermal motion slows and (2) out-of-focus thermal motion is relatively rare, once every 5-20 seconds. Thus, the capsids are visualized in real time while executing thermal motion that is both slowed and effectively two-dimensional. Both dissociation and formation of phage capsid dimers have been observed in real time (Wang et al., J. Microsc. 2005, 217:83-92). If adherence to either glass or gel limits results, adherence can be inhibited with anti-adherence agents, such as gelatin, bovine serum albumin and non-ionic detergents. Neither non-ionic nor ionic detergents detectably damage either phage or MLD capsid II.

Chemistry. With this background, the following strategy is used for producing MLD capsid II that contains dyes sealed stably enough so that they do not leave MLD capsid II unless in extreme conditions. The dyes are proxies for drugs of similar mw and electrical charge. It is noted that not all applications require that a dye/drug be “unloaded” after delivery, because some dyes/drugs can be made to kill cells (and viruses) photodynamically, by release of reactive oxygen species, when exposed to visible light. Reactive oxygen is expected to diffuse through the shell of MLD capsid II and damage cells to which MLD capsid II either is attached or has entered. In the past, proflavine has been painted on skin lesions (no delivery vehicle) and used as an anti-viral via photodynamic action (O'Day et al., Am. J. Ophthalmol. 1975, 79:941-948). Porphyrins, via use of their precursors as the drug, have been similarly used as a cancer therapeutic (Kaur et al., J. Nanotechnol. 2012, Article ID 247427).

In certain aspects (1) the thermal motion (diffusion)-driven entry of dye into MLD capsid II (loading) is optimized and (2) then the dye is trapped in the internal cavity of MLD capsid II (sealing), so that dye molecules will not exit until MLD capsid II encounters conditions appropriate for its use as a delivery vehicle. To optimize loading, the loading rate is determined at various temperatures, pH values and ionic strengths, for example, vs. the molecular weight and electrical charge of the compound loaded. In certain aspects this can be done via fluorescence (FIG. 5) or fluorescence quenching of a dye already loaded (FIG. 6). Based on previous experience with phage T4 (Griess et al., Biopolymers 1991, 31:11-21), the loading rate increases as temperature increases. MLD capsid II is stable to 80° C. Thus, MLD capsids can be loaded at temperatures up to 80° C. In certain aspects capsids can be loaded at 20, 30, 40, 50, 60, 70, or 80° C., including all values and ranges there between.

As the temperature decreases, the loading rate is expected to decrease, eventually to zero at a critical temperature. The critical temperature is expected to decrease as the molecular weight of the compound loaded decreases. The borderline for loading MLD capsid II at 25° C. is somewhat higher than 612, based on the fact that iothalamate anion (mw =612) loads, but very slowly (>19 hours), as determined by the buoyant density after incubation with sodium iothalamate. That is to say, one is able to seal a compound by lowering the temperature. The highest non-lethal body temperature is about 42° C. So, the initial objective is to have a seal that is stable to about 47° C., i.e., with a safety margin of 5° C.

In certain aspects MLD capsid II is used to load (at >47° C.) and seal (at >42° C.) anti-cancer compounds with mw close to that of Nycodenz (Nycodenz mw=821). These compounds include microtubule assembly-inhibiting drugs, vinblastine (mw=811), vinfluine (mw=817) and taxol (mw=854). Success is favored by the following observation made by AUC-BD in a Nycodenz density gradient. Nycodenz does not enter MLD capsid II after pre-centrifugation incubation at temperatures as high as 54° C. But, it does enter after pre-centrifugation incubation at 61° C. and 68° C., as judged by conversion of MLD capsid II to a high-density particle without changed peak width; native gel electrophoresis confirms shell intactness (unpublished).

For a compound that has entered and been sealed in MLD capsid II, the tightness of the seal can be determined by conducting AUC-BD in both the presence and the absence of the compound at several temperatures. If the compound slowly leaks from MLD capsid II, the density in the absence of the compound will progressively decrease as AUC-BD proceeds, while the density in the presence of the compound will not change. The AUC-BD will also determine whether heterogeneity exists in the loading of MLD capsid II particles. Heterogeneity of loading will cause heterogeneity of density. For each compound, loading and sealing based on AUC-BD determines the kinetics of loading via fluorescence procedures (see FIGS. 5 and 6).

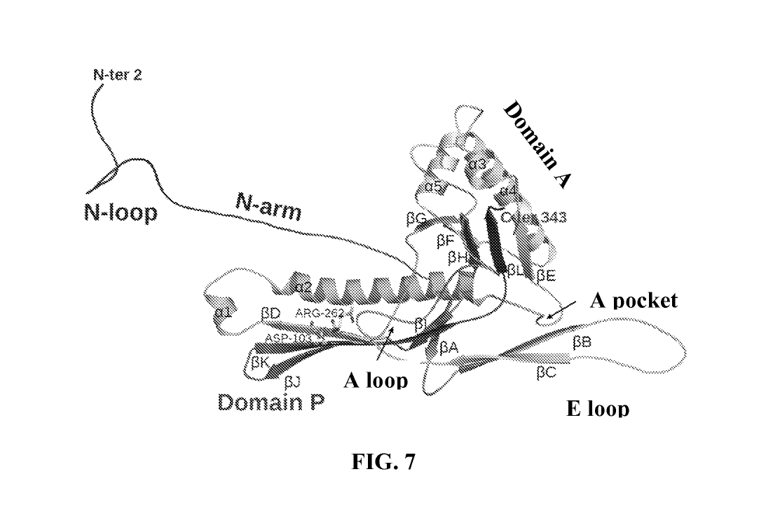

For each compound with AUC-BD-derived evidence of loading/sealing, the AUC-BD observations are checked for accuracy in conditions more resembling those in which the MLD capsid II vehicles will be used, i.e., at atmospheric pressure and in the absence of Nycodenz. This can be done by determining single-capsid values of I, by use of single-molecule fluorescence microscopy (see FIG. 7). The single MLD capsid II particles can be observed by staining them with the red fluorescence of Alexa Fluor 594. The entry of compounds will be separately observed via color-based image splitting, in a bis-ANS fluorescence-quenching mode (FIG. 6), for those compounds that quench the fluorescence of MLD capsid II-bound bis-ANS. The procedure of specimen preparation can be the “confining of buffer” procedure previously developed (Wang et al., J. Microsc. 2005, 217:83-92; Wang et al., J. Microsc. 2004, 213:101-109; Gai et al., J. Microsc. 2007, 226:256-262).

To accelerate the finding of conditions for loading and sealing, the following systematic study can be performed for a variety of drugs and dyes with molecular weight between ˜300 and ˜1,000. The rate of entry into MLD capsid II vs. compound molecular weight, electrical charge and shape is determined. MLD capsid II is incubated in the presence of a compound and measurement is made of either (1) I (fluorescence intensity), in the case of a compound that undergoes an increase in I during binding to the interior of MLD capsid II, bis-ANS (mw=597; charge=−2), for example (FIG. 5); or (2) quenching of the/of pre-bound bis-ANS, in the case of a compound that quenches (FIG. 6). The quenching compounds include, but are not limited to, ethidium (mw=314; charge, d+1), ethidium homodimer (mw=716; charge=+2), iothalamate (mw=612; charge =−1) and additional compounds in the molecular weight range between 300 and 1,000, including anti-cancer compounds with a positive charge, such as DNA—or membrane-binding drugs, cisplatin (cationic mw=807, charge, +1), carboplatin (cationic mw=371; charge,+2), daunomycin (cationic mw=528; charge, +1) and other anthracycline antibiotics. These drugs, like the positively charged ethidium cation, should bind and quench the fluorescence of (negatively charged) bis-ANS that is bound to the interior of MLD capsid II.

In these studies, the rate of entry may not correlate precisely with molecular weight, if either molecule shape or molecular charge varies. One can determine whether non-correlation of this type occurs.

Genetics. Genetics can be used to make changes in MLD capsid II that are needed to achieve success in loading and sealing. For example, as compounds become larger, eventually one will reach a point such that temperatures needed to induce entry will be high enough to cause disruption of MLD capsid II. In response, one can mutate the connector so that the most constricted part of its axial channel is larger at elevated, sub-disruption temperature, but can still be sealed at a lowered temperature.

Cryo-EM-based information about connector structure can be used to optimize the mutagenesis. The initial mutagenesis will be performed by error-prone PCR of the region of gene 8 that encodes the gp8 segment that protrudes into the channel of the connector. This region is called the tunnel loop (FIG. 2). The mutagenized gene 8 PCR fragment is incorporated in a wild type genome, thereby generating mutants in the targeted region of gene 8.

Next, one selects among the mutagenized phages for temperature sensitive (ts) mutants. A ts mutant is a mutant that forms plaques at relatively low temperature (typically 30° C.), but does not grow at higher temperature (typically 40° C.). The ts mutants will be highly enriched (if not 100% enriched) in mutations of the region of gene 8 that encodes the tunnel loop, because of the selective mutagenesis. Thus, these mutants should be highly enriched for mutants that produce MLD capsid II with a channel that has an altered permeability, in many cases with temperature sensitivity of loading/sealing greater than that of the wild type MLD capsid II. To extend the range of mutants, one may also select for cold-sensitive (cs) mutants. A cs mutant does not grow at relatively low temperature, typically 25° C., although it does grow at a higher temperature, typically 30° C.

In certain aspects one can screen for ts and cs mutants that generate MLD capsid II that has a connector channel permeable enough and temperature sensitive enough to allow the elevated temperature-dependent loading and lowered temperature-dependent sealing of compounds larger than those that can be loaded and sealed via temperature changes with the wild type MLD capsid II. To do this, one isolates the MLD capsid II of each ts and cs mutant and, for each MLD capsid II, one tests permeability during loading and sealing, by use of the procedures described herein. One can also screen for mutants that produce MLD capsid II that has improved loading and sealing characteristics for the smaller compounds.

At the conclusion of the work one has mutant MLD capsid II's that fall into several classes. Each class will be characterized by a narrow range of loading and sealing temperatures for compounds of given molecular weights and charges. Based on rates of entry of compounds of known characteristics, one will simplify this classification by deriving an effective pore radius at each temperature, in analogy with what was previously done to determine the radius of the effective pore of gels vs. both gel concentration and conditions of gelation (Griess et al., Biopolymers 1989, 28, 1475-1484). Thus, one can combine this gene with any mutant gene 10 that will be generated, i.e., during selection for specificity.

Rapid, mutation/selection can be used to generate shells that specifically bind to any chosen target or cellular surface.

The gp10 shell. The gp10 subunits of the shell of both T3/T7 phage and MLD capsid II are arranged with the C-terminus of gp10 exposed at the exterior surface. Thus, inserts in the C-terminus-encoding region of gene 10 are used for peptide display. Cryo-EM with symmetric 3D reconstruction was performed to form 3.2 Å reconstructions of entire shells. These reconstructions include 3.2 Å details of the gp10 subunits of both T3 and T7 phage and MLD capsid II. From these reconstructions, it is know how the C-terminus of gp10 projects to the outside. One also knows that the N-terminus moves from the inside of the shell to the outside during DNA packaging, but does not occupy as much surface as the C-terminus in the mature phage. That is to say, the tip of the N-terminus is also on the phage and MLD capsid II surface.

To illustrate the details for both the C-terminus and the rest of gp10, a single subunit of MLD capsid II-associated T7 gp10 was separated from a symmetrized cryo-EM reconstruction performed by the laboratory of Wen Jiang, Purdue University with MLD capsid II from the Serwer laboratory. The reconstruction and various domains of assembled gp10 are shown in FIG. 7. The outside surface of the shell is at the top, the inside surface, at the bottom. Three alpha-helical regions and an unstructured loop of the C-terminal domain (domain A; FIG. 7) are exterior surface-exposed and are in position to determine binding properties of the capsid. The conformation of gp10 is in the HK97-class (Veesler et al., Structure 2012, 20:1384-1390).

Selective Mutagenesis by PCR: The gp10 shell. Biologically, the tail and tail fibers of all phages are major factors in determining host specificity (reviewed in Casjens and Molineux, Adv. Exp. Med. Biol. 2012, 726:143-179). However, evidence exists that the shell is also involved. Specifically, a quantized increase in the magnitude of the (negative) surface electrical charge density (σ) of the gp10 shell is associated with converting T7 phage from host-binding to host-non-binding (Gabashvili et al., J. Mol. Biol. 1997, 273:658-667). The evidence also suggests that this quantized surface charge switching was evolutionarily selected to prevent the phage from being inactivated by binding to non-host surfaces. The assay for binding was centrifugal pelleting at speed sufficient to pellet bacterial cells, but not phage particles; unabsorbed phages were detected in the supernatant by either plaque assay or native gel electrophoresis. Thus, binding specificity for the mature phage can apparently be generated via the shell, as well as the tail.

Directed mutagenesis is a procedure that can be used to generate changes in binding specificity and to direct the site of the changes toward shell protein, gp10. That is to say, if one specifically mutates the surface-exposed region of gp10 and then selects for mutants with a desired binding specificity, most (possibly all) of the specific binding-promoting mutations will produce an altered gp10 shell, not an altered tail.

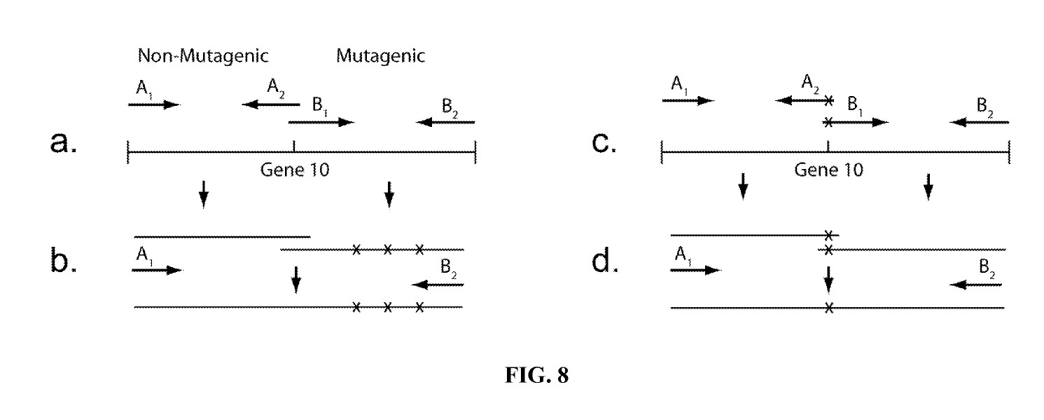

The inventor's laboratory performed site-specific, primer-specified mutagenesis of gene 10, as illustrated in in FIG. 11c. This was done by introducing the same mutation in two overlapping primers (A₂ and B₁ in FIG. 8c), used to generate two overlapping PCR fragments, as illustrate in FIGS. 8c and 8d. To transfer the mutation to a complete genome, PCR fusion was performed, by use of the A₁ and the B₂ primers (FIG. 8d), to create a mutagenized fragment with sufficient homology to subsequently incorporate by use of genetic recombination. The fusion depends on partial overlap of the A₂ and B₁ primers in FIG. 8c.

The following procedure was then used to transfer the mutation to a complete phage genome. The PCR fusion product of FIG. 8d was cloned, via restriction sites introduced in the A₁ and B₂ primers. The cloning vector was a commercial plasmid-based T7 expression vector (DE3) in an E. coli BL21 host, i.e., E. coli BL21(DE3) (Daegelen et al., J. Mol. Biol. 2009, 394:634-643). The vector was infected with T7 phage amber mutant in gene 10 and screened for mutants among the progeny plated on a host non-permissive for amber mutants. The use of a gene 10 amber mutant and a non-permissive host caused selection for progeny phages that have recombined in vivo with the cloned, mutagenized PCR fragment. Therefore, the progeny phages had a high probability of having incorporated the primer-engineered mutation.

For increasing efficiency of mutagenesis, an interesting alternative mutation-transfer/incorporation procedure is suggested by bypassing the use of cloning in the formation of T3/T7 genomic hybrids (Khan et al., Virology 1997, 227:409-419). Cloning was bypassed by using in vitro recombination of PCR fragment and amber mutant genome directly in an extract of T7-infected cells. The extract conducted both recombination and, then, packaging to introduce the recombined (hybrid) genome to an infective phage particle.

In preliminary, unpublished work, the procedure of FIGS. 8c and 8d has been implemented. Cloning in E. coli BL21(DE3) was used and in vivo recombination to produce a mutant with two specific (primer-engineered) mutations in the N-terminus of T3 gp10, Q8A/Q9A.

Mutagenesis optimized for subsequent selection. Anticipating that rapid, inexpensive changing of specificity is needed, a system can be developed for specificity selection that is as rapid. This system will be based on the related phages, T3 and T7, which are the most rapidly reproducing, known phages. T3 and T7 produce plaques in only 2.5-3.0 hours at 37° C. Thus, whenever propagation is needed, the time of propagation is not a significant factor. Production of mutagenized phage inocula and subsequent selection and associated propagation will take days, not the weeks or months needed with either cellular (antibody) or other viral systems. To initiate the first selections, we will use phages that have been randomly mutagenized in a selected region of gene 10. More precise mutagenesis can be used as more is known about what the most appropriate targets for mutagenesis are.

To perform random mutagenesis of a selected region, for example, the C-terminus-encoding region of gene 10, this region is amplified by use of mutagenic PCR (Matsumura and Rowe, Biomol. Eng. 2005, 22:73-79; Kumar et al., Protein Eng. Des. Sel. 2006, 19:547-554). The B₁ and B₂ primers in FIG. 8a illustrate the primers used for this amplification. PCR fusion (FIG. 8b) is then used to join each mutagenized PCR fragment to an un-mutagenized, overlapping PCR fragment (A₁ and A₂ primers at the left in FIG. 8a). Again, the fusion increases the region of homology and, therefore, increases the probability of initiating recombination during transfer to a mature genome. The various mutant fusion products are cloned in an E. coli BL21(DE3) vector to form a library of mutants of gene 10. E. coli BL21(DE3) is non-permissive for amber mutants. Thus, a gene 10 amber mutant phage can propagate on this host only if it either reverts or recombines with gene 10 cloned in the DE3 vector.

It is contemplated that, after cloning, the fusion product of FIGS. 8a and 8b has sufficient homology to be transferred from plasmid cloning vector to phage genome by in vivo T7 recombination. The inventor anticipates this based on the homology required in previous work both with in vitro recombination-based hybrid phage formation and independently with in vivo recombination-based mutagenesis at the N-terminus-encoding region of gene 10 via the procedure in FIGS. 8c and 8d. The recombination frequency of T7 is unusually high, so high that the original genetic map had to be constructed in relatively short segments (Studier Virology 1969, 39:562-574). However, if homology is not sufficient, an additional homologous region is added by performing a second fusion with a partially overlapping, un-mutagenized PCR fragment that extends to the right of gp10 in FIG. 8a.

To produce a phage inoculum for selection for shell binding specificity, the following is done. Phage (either T3 or T7) amber mutant in gene 10is plated on E. coli BL21(DE3) cells with a mutant gene 10 library for that phage. Plating is at high density (1,000-5,000 plaques per Petri plate). Progeny phages are gathered from the plate and debris pelleted to make a phage preparation (i.e., the traditional plate stock). As discussed above, viable phages will be primarily those that have lost the amber mutation during recombination with cloned gene 10. This recombination will also transfer the various mutations and produce a mutant phage library. The reversion background of amber mutants is 1:10⁵ -1:10⁶. Thus, even with recombination frequency as low as 1:10⁴, adequate discrimination exists between recombination and reversion.

Using T7 one can, if needed, display peptides appropriate to further optimize chances of success in subsequent selection for binding specificity. For example, if one wants to further select for phages that have received a mutagenized PCR fragment, a His-tag with linker can be added to the A₂ and B₁ primers of FIG. 8a, so that phages receiving a mutagenized PCR fragment will also receive a His-tag. The mutagenized phages are purified by binding to and eluting from a nickel column. T7 capsid concentrations have been increased for cryo-EM by His-tagging a wild type T7 phage and adsorbing phage and capsids in an unfractionated lysate to a nickel-derivatized support substrate for cryo-EM (Serwer, P. and Jiang, W. Bacteriophage 2012, 2, 239-255.).

In an effort to accelerate and simplify the production of mutagenized inocula for selection, various procedures for mutagenized fragment incorporation can be pursued. A procedure to be explored is the use of in vitro recombination (rather than in vivo recombination) of fused, mutagenized PCR fragment with mature DNA amber mutant in gene 10. This in vitro procedure is basically equivalent to the in vivo procedure, but it is also simpler and more rapid, although not yet tested to the extent that the in vivo procedure has been tested.

The overall procedure of mutagenesis is optimized by optimizing the level of mutagenic PCR. If information is sufficient to introduce specific (rather than random) mutations before selection, specific mutations can be introduced via a variation of the procedure in FIGS. 8c and 8d.

Selection for binding specificity of phage and MLD capsid II to test cells: Bacteria. T3 and T7 (gene 10) phage mutants are selected that preferentially bind to one bacterial cell (binder cell), but not another bacterial cell (non-binder cell). In a first study, phages mutated in either the C-terminus-encoding A (surface) domain of gene 10 or the extreme N-terminal (surface) region of gene 10 are used. A PCR-mutagenized inoculum is the starting material. Additional plate stock propagations are interspersed among selections when levels of phage become too low to continue selection. In at least some cases, the needed binding specificity will require multiple mutations, some of which may not be in the original, PCR mutagenized inoculum. Non-gene 10 mutations should be rare in the final product because (1) gp10 is the major surface protein of the T3 and T7 shells and (2) selection does not require tail function. If needed to further improve selectivity, mutagenesis of the selected region of gene 10 can be repeated after extracting DNA from surviving phage from a previous selection.

Independently, the level of PCR mutagenesis is systematically varied before selection, to determine the level of mutagenesis that optimizes the selection. The outcome of any given mutagenesis protocol is determined by determining the number of selections needed to achieve a given level of binding specificity.

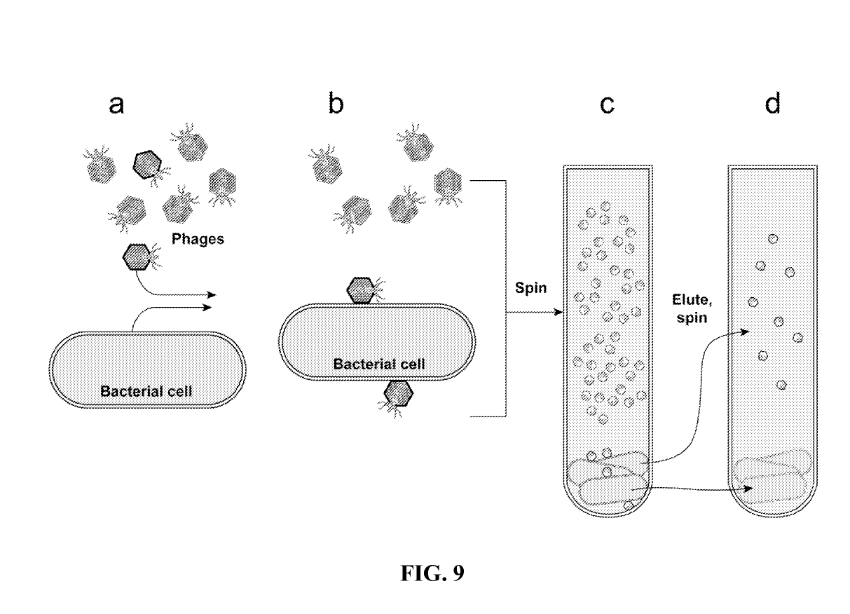

One can use the following selection, illustrated in FIG. 9. (1) mutated phages are incubated with the binder cells (FIG. 9a). (2) After binding (FIG. 9b), the binder cells are pelleted with adsorbed phage (FIG. 9c); the pellet is saved and wash/pellet again. (3) phages are eluted from binder cells by use of one of the following procedures, followed by pelleting the cells (FIG. 9d) and keeping the supernatant of eluted phages (all phages are shown the supernatant in FIG. 9d): raising ionic strength, lowering ionic strength, raising pH, lowering pH, changing the composition of the solution. Exposure to binder cells will be done in conditions that mimic conditions that occur when MLD capsid II is used as a drug delivery vehicle.

Finally, the eluted phages are exposed to non-binder cells using the conditions previously used for exposure to binder cells. Then, phages will be selected for non-binding by pelleting the cells and retaining the supernatant represented in FIG. 9c, rather than the pellet. The supernatant will be enriched for phage that now do not bind the non-binder cells, but previously bound the binder cells. This supernatant will be used for another cycle of bind/non-bind selection. The total process will encompass several cycles of selection for phages that bind the binder cells, but not the non-binder cells.

Two selection cycles per day can be run, manually. If each selection is, conservatively, 50% efficient in enriching for desired binding characteristics, enrichment by 16× will occur after only two days. If only 1% of the mutants have the binding characteristic needed, less than a week of selection will be needed to obtain a pure clone of each of these mutants. In the future, automation will provide the opportunity for round-the-clock selection. The expectation is that the selection will become increasingly efficient as experience is gained, thereby either further reducing time or lowering the threshold for the production of the desired mutants during mutagenesis.

To test the effectiveness of the selection, every two cycles, determination will be made of the percentage of viable phages that binds binder cells and the percentage that does not bind non-binder cells. The ratio of these two percentages will be defined as the specificity. The binding will be determined by either plaque assay or native gel electrophoresis before and after exposure to and pelleting of cells. As the specificity rises to over ˜50, the selection will be terminated, and phages will be 3× single-plaque purified from the final mixture. Binding specificity of the purified phages will then be similarly determined.

Purification of evolved MLD capsid II for testing of effectiveness. The next step is to purify MLD capsid II produced by a mutated/selected phage with high binding specificity. This is done by, first, preparing a lysate of E. coli cells infected by a 3× plaque-purified version of this phage. One then polyethylene glycol-precipitate/concentrates MLD capsid II and purifies it, starting with centrifugation in cesium chloride density gradients. The next and final step is buoyant density centrifugation in a density gradient of either Metrizamide or the more recently developed, closely related compound, Nycodenz. The entire process now takes about four days. MLD capsid II has a density (1.086 g/ml) that is less than the density of any other particle that has been found in a lysate of phage-infected E. coli. One reasonably expects that the mutated/selected MLD capsid II will have the same loading and sealing characteristics as the MLD capsid II of the phage before selection for binding specificity. The reason is that the binding selection focuses on the region of gp10 on the external surface of the shell, whereas the permeability is determined primarily by gp8 and secondarily by gp10-gp 10 contacts.

The loading and sealing characteristics of the mutant MLD capsid II can be tested. To do this, one will use the procedures of AUC-BD, fluorescence binding kinetics, and single-particle fluorescence microscopy previously described. If the permeability characteristics have significantly changed, these characteristics can be re-optimized. We will then load the evolved MLD capsid II with either drugs or dyes (some dyes are drugs) for tests. One will determine the extent to which the MLD capsid II particles vary in loading by use of both AUC-BD and single-particle fluorescence microscopy. The fluorescence microscopy will also determine the extent to which particles are aggregated. Aggregation of MLD capsid II, if it occurs, presumably will reduce effectiveness.

If aggregation occurs, then one or more the following steps can be taken to prevent aggregation. (1) hydrophilic amino acids can be added to the C-terminus before selection. (2) MLD capsid II from a third related phage, phi II, can be used, which has a surface that is more hydrophilic, based on a negative o that is higher than it is for the MLD capsid II of phages T3 and T7. (Serwer, P. et al. J. Mol. Biol. 1983, 170, 447-469.) (3) non-aggregation can be selected during the selection for binding specificity.

The use of a model test system. Tests can be performed that use drug/dye-loaded MLD capsid II to manage model binder cells. Both binder and non-binder cells will be chosen for efficiency (in time and cost) of the testing. In this example the model cells will be bacterial cells.

Drugs that can be loaded in drug delivery vehicles described include, but are not limited to chlorhexidine, mw=505; nystatin, mw=926; amphotericin b, mw=924, and drugs with similar molecular weights.

Managing of bacterial cells. The following tests can be used to determine the effectiveness of specific MLD capsid II vehicles in managing the propagation of bacterial cells. First, it will be determined how long dyes remain sealed in MLD capsid II. The MLD capsid II will be incubated at several temperatures and under buffer conditions similar to those to be encountered in managing bacterial cells. The retention of either a drug or a dye will be determined by use of either AUC-BD or fluorescence.

For those evolved MLD capsid II vehicles that can be well sealed after being loaded, the capacity of the vehicle to manage the propagation of binder bacteria can be tested. In initial test, a drop of fluorescent dye (proflavine, for example)-loaded, high specificity MLD capsid II can be placed on two agar-supported lawns of binder bacteria in a Petri plate. One lawn will be exposed to visible, fluorescent light; the other will be kept in the dark. The multiplicity will initially be between 2 and 10 MLD capsid II particles per bacterial cell. If the loaded MLD capsid II inhibits propagation of the binder cells, then a partial clearing will be observed where the loaded, evolved MLD capsid II was spotted, for both plates. If photodynamic action is required, then the clearing will be observed only for the plate exposed to light.

The source of any clearing observed will be determined via performing of the following controls. (1) Use of an unloaded version of the same vehicle will determine whether any clearing observed was caused by the vehicle alone (clearing also observed in this case) or by effects that depend on presence of the load (no clearing observed in this case). (2) Use of MLD capsid II that has either not been mutated/selected or has been selected for an alternative specificity will determine whether the any observed clearing requires the selected specificity (no clearing in this case) or not (clearing in this case). If clearing is dependent on both the load and a mutated/selected version of MLD capsid II, then a precursor for the managing of bacterial and fungal infections has been identified. This procedure can be used as a basic procedure for managing neoplasms. Utility will be especially high for infection (and neoplasms) in places accessible to visible light, i.e., on skin and just beneath skin (the uvea, example). The use of photodynamic action further increases the specificity via the use of narrow beams of light.

Successful photodynamic treatment of skin infections has, in the past, been successful. With a specific delivery vehicle, the total amount of dye can be lowered and the dye's access to healthy cells is suppressed. Photodynamic anti-microbial therapy would, thus, be less hazardous if a specific, loaded vehicle were used.

To enhance the effectiveness of loaded, specific MLD capsid II vehicles, capsids will be modified with peptide that enhances bacterial cells uptake. Positively charged, “Trojan horse” peptides can be added to gp10, either at the C-terminus or, if needed to prevent interference with specificity, elsewhere in the surface-exposed region of gp10, the extreme N-terminus-encoding region. These peptides can enhance the uptake by both prokaryotic and eukaryotic cells (Lindberg et al., Therapeutic Delivery 2:71-82, 2011; Khafagy et al., Advanced Drug Delivery Rev. 64:531-539, 2012).

The specificity of loaded, specific MLD capsid II vehicles can be determined by the extent to which bacteria can be specifically eliminated from liquid cultures that have a mixture of both binder and non-binder bacteria. Evolved, loaded MLD capsid II vehicle can be added at a multiplicity greater than 1 to a mixed culture in log phase. The mixed culture is incubated until stationary phase and the ratio of binder to non-binder cells determined at various stages in the incubation.

The above compositions can be administered using conventional modes of delivery including, but not limited to, intravenous, intraperitoneal, oral, intralymphatic, subcutaneous administration, intraarterial, intramuscular, intrapleural, intrathecal, and by perfusion through a regional catheter. Local administration to a tumor in question is also contemplated by the present invention. When administering the compositions by injection, the administration may be by continuous infusion or by single or multiple boluses. For parenteral administration, the anti-metastatic agents may be administered in a pyrogen-free, parenterally acceptable aqueous solution comprising the desired anti-cancer agents in a pharmaceutically acceptable vehicle. A particularly suitable vehicle for parenteral injection is sterile distilled water in which an one or more anti-cancer agents are formulated as a sterile, isotonic solution, properly preserved. MLD capsid II is not unstable in any known aqueous solution.

If desired, stabilizers that are conventionally employed in pharmaceutical compositions, such as sucrose, trehalose, or glycine, may be used. Typically, such stabilizers will be added in minor amounts ranging from, for example, about 0.1% to about 0.5% (w/v). Surfactant stabilizers, such as TWEEN®-20 or TWEEN®-80 (ICI Americas, Inc., Bridgewater, N.J., USA), may also be added in conventional amounts.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents. Compositions for parental administration are also sterile, substantially isotonic and made under GMP conditions.

For the compounds of the present invention, alone or as part of a pharmaceutical composition, such doses are between about 0.001 mg/kg and 1 mg/kg body weight, preferably between about 1 and 100 ug/kg body weight, most preferably between 1 and 10 ug/kg body weight. In other aspects a pharmaceutical composition may include 1×10⁵, 1×10⁶, 1×10⁷, 1×10⁸, 1×10⁹, 1×¹⁰, or more drug delivery vehicles.

Therapeutically effective doses will be easily determined by one of skill in the art and will depend on the severity and course of the disease, the patient's health and response to treatment, the patient's age, weight, height, sex, previous medical history and the judgment of the treating physician.

In some methods of the invention, the cancer cell is a tumor cell. The cancer cell may be in a patient. The patient may have a solid tumor. In such cases, embodiments may further involve performing surgery on the patient, such as by resecting all or part of the tumor. Compositions may be administered to the patient before, after, or at the same time as surgery. In additional embodiments, patients may also be administered directly, endoscopically, intratracheally, intratumorally, intravenously, intralesionally, intramuscularly, intraperitoneally, regionally, percutaneously, topically, intrarterially, intravesically, or subcutaneously. Therapeutic compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

Methods of treating cancer may further include administering to the patient chemotherapy loaded in a capsid vehicle described herein, which may be administered more than one time. Chemotherapy includes, but is not limited to, cisplatin (CDDP), carboplatin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, taxotere, taxol, transplatinum, 5-fluorouracil, vincristin, vinblastin, methotrexate, gemcitabine, oxaliplatin, irinotecan, topotecan, or any analog or derivative variant thereof.

In some embodiments, the cancer that is administered the composition(s) described herein may be a bladder, blood, bone, bone marrow, brain, breast, colorectal, esophagus, gastrointestine, head, kidney, liver, lung, nasopharynx, neck, ovary, pancreas, prostate, skin, stomach, testicular, tongue, or uterus cell. In certain aspects the cancer is melanoma or breast cancer.

1. A drug delivery vehicle comprising a capsid shell/connector from a double stranded DNA virus with a drug.

2. The composition of claim 1, wherein the capsid shell comprises gp10 proteins and gp8 proteins.

3. The composition of claim 2, wherein the gp10 and gp8 proteins are phage T3 or phage T7 proteins.

4. The composition of claim 1, wherein the drug is a chemotherapeutic or antibiotic drug.

5. The composition of claim 1, further comprising a modified gp10 that selectively binds a target.

6. The composition of claim 5, wherein the target is a bacteria or eukaryotic cell with an aberrant phenotype.

7. The composition of claim 6, wherein the eukaryotic cell is a cancer cell.

8. A method for loading a capsid shell comprising:
(i) contacting a solution comprising a drug to be loaded with a solution comprising a gp10 and gp8 proteins at a temperature that facilitates loading of the capsid shell; and
(ii) lowering the temperature of the drug/capsid solution to a level that seals the drug in a capsid shell.
